(19) United States
(12) Patent Application Publication
(10) Pub. No.: US 2004/0192593 A1
(43) Pub. Date: Sep. 30, 2004

(54) Protease resistant ti-growth hormone releasing hormone

(75) Inventors: Ruxandra Draghia-Akli, Houston, TX (US); Marta L. Fiorotto, Houston, TX (US); George Taffet, Houston, TX (US)

Correspondence Address: Jackson Walker L.L.P., Suite 600, 2435 N. Central Expressway, Richardson, TX, 75080, US

(73) Assignee: Baylor College of Medicine, Houston, TX

(21) Appl. No.: 10/166,356

(22) Filed: Aug. 21, 2002

Related U.S. Application Data
(63) Continuation-in-part of application No. 09/624,268, filed on Jul. 24, 2000, now Pat. No. 6,551,996.
(60) Provisional application No. 60/145,624, filed on Jul. 26, 1999.

(51) Int. Cl. A61K 38/17

(52) U.S. Cl. 514/12

(57) ABSTRACT

One aspect of the current invention is a composition for a modified growth hormone releasing hormone (“GHRH”) or functional biological equivalent thereof. Another aspect of the current invention includes a nucleic acid molecule that encodes the modified GHRH or functional biological equivalent. The modified GHRH can be defined as a biologically active polypeptide that was engineered to contain a distinct amino acid sequence while simultaneously having similar or improved biologically activity when compared to a wild-type GHRH (“wt-GHRH”) polypeptide. Another aspect of the current invention includes a method for delivering the composition of this invention to a subject, wherein the modified GHRH increases the level of growth hormone (“GH”) secretion in a subject. The preferred subject is a human or domesticated animal. Additionally, the modified GHRH composition is resistant to degradation when compared to the wt-GHRH.

### RELATED APPLICATIONS

This application is a continuation-in-part of the U.S. patent application, Ser. No. 09/624,268, entitled “Super-Active Porcine Growth Hormone Releasing Hormone Analog,” filed on Jul. 24, 2000. The Ser. No. 09/624,268 application claims priority to U.S. Provisional Patent Application, Serial No. 60/145,624, entitled “Super-Active Porcine Growth Hormone Releasing Hormone Analog,” filed on Jul. 26, 1999, the entire content of each of which is hereby incorporated by reference.

### BACKGROUND

One aspect of the current invention is a composition for a modified growth hormone releasing hormone (“GHRH”) molecule or functional biological equivalent thereof. The composition may also be a nucleic acid molecule that encodes the modified growth hormone releasing hormone (“GHRH”) or functional biological equivalent thereof. The modified GHRH can be defined as a biologically active polypeptide that has been engineered to contain a distinct amino acid sequence while simultaneously having similar or improved biologically activity when compared to a wild-type GHRH (“wt-GIIRII”) polypeptide. One benefit of the claimed invention occurs when the modified molecule with the GHRH composition is delivered to a subject. The modified GHRH increases the level of growth hormone (“GH”) secretion in a subject. Other benefits outlined in preferred embodiments include: increased insulin-like growth factor I (“IGF-I”), increased weight gain; increased bone density, increases body length, improved immune function and improved cardiac function in aging mammals. The modified GHRH composition of this invention is also resistant to degradation compared to the wt-GHRH.

Regulated expression of the growth hormone (“GH”) pathway is essential for optimal linear growth, as well as homeostasis of carbohydrate, protein, and fat metabolism. GH synthesis and its pulsatile secretion from the anterior pituitary is stimulated by growth hormone releasing hormone (“GHRH”) and inhibited by somatostatin, both hypothalamic hormones (Frohman et al., 1992). GH increases production of insulin-like growth factor-I (IGF-I) primarily in the liver, as well as other target organs. IGF-I and GH feedback on the hypothalamus and pituitary to inhibit GHRH release and GH secretion. The endogenous rhythm of GH secretion becomes entrained to the imposed rhythm of exogenous GHRH (Caroni and Schneider, 1994).

Linear growth velocity and body composition respond to GH or GHRH replacement therapies in a broad spectrum of conditions, both in humans and in farm animals. The etiology of these conditions can vary significantly. In 50% of human GH deficiencies the GHRH-GH-IGF-I axis is functionally intact, but does not elicit the appropriate biological responses in its target tissues. Similar phenotypes are produced by genetic defects at different points along the GH axis (Parks et al., 1995), as well as in non-GH-deficient short stature. In the non-GH-deficiency causes of short stature, such as Turner syndrome (Butler et al., 1994), hypochondroplasia (Foncea et al., 1997), Crohn's disease (Parrizas and LeRoith, 1997), intrauterine growth retardation (Hoess and Abremski, 1985) or chronic renal insufficiency (Lowe, Jr. et al., 1989), GHRH or GH therapy can be effective in promoting linear growth (Gesundheit and Alexander, 1995). In the elderly, the GHRH-GH-IGF-I axis undergoes considerable decrement, with reduced GH secretion and IGF-I production associated with a loss of skeletal muscle mass (sarcopenia), osteoporosis, increased fat deposition and decreased lean body mass (Caroni and Schneider, 1994; Veldhuis et al., 1997). It has been demonstrated that the development of these changes can be offset by recombinant GH therapy. GH replacement therapy both in children and the elderly is widely used clinically. Current GH therapy has several shortcomings, however, including frequent subcutaneous or intravenous injections, insulin resistance and impaired glucose tolerance (Rabinovsky et al., 1992); children are also vulnerable to premature epiphyseal closure and slippage of the capital femoral epiphysis (Liu and LeRoith, 1999). A “slow-release” form of GH (Genentech), which requires injections every 14 days, perturbs the normal physiological pulsatile GH profile, and is also associated with frequent side effects.

In domestic livestock, GHRH and GH stimulate milk production, with an increase in feed to milk conversion, which additionally enhances growth, primarily by increasing lean body mass (Lapierre et al., 1991; van Rooij et al., 2000) with overall improvement in feed efficiency. Hot and chilled carcass weights are increased and carcass lipid (percent of soft-tissue mass) is decrease by GHRH and GH (Etherton et al., 1986).

Administering novel GHRH analog proteins (U.S. Pat. Nos. 5,847,066; 5846,936; 5,792,747; 5,776,901; 5,696,089; 5,486,505; 5,137,872; 5,084,442, 5,036,045; 5,023,322; 4,839,344; 4,410,512, RE33,699), synthetic or naturally occurring peptide fragments of GHRH (U.S. Pat. Nos. 4,833,166; 4,228,158; 4,228,156; 4,226,857; 4,224,316; 4,223,021; 4,223,020; 4,223,019) for the purpose of increasing release of growth hormone have been reported. GHRH analogs containing the following mutations have been reported (U.S. Pat. No. 5,846,936): Tyr at position 1 to His; Ala at position 2 to Val, Leu, or others; Asn at position 8 to Gln, Ser, or Thr; Gly at position 15 to Ala or Leu; Met at position 27 to Nle or Leu; and Ser at position 28 to Asn. The GHRH analog is the subject of U.S. patent application Ser. No. 09/624,268 (“the '268 patent”), which teaches application of a GHRH analog containing mutations that improve the ability to elicit the release of growth hormone. In addition, the '268 patent relates to the treatment of growth deficiencies; the improvement of growth performance; the stimulation of production of growth hormone in an animal at a greater level than that associated with normal growth; and the enhancement of growth utilizing the administration of growth hormone releasing hormone analog and is herein incorporated by reference.

Although GHRH protein therapy stimulates normal cyclical GH secretion with virtually no side effects (Corpas et al., 1993), the short half-life of the molecule in vivo requires frequent (one to three times per day) intravenous, subcutaneous or intranasal (at 300-fold higher dose) administrations. Thus, recombinant GHRH administration is not practical as a chronic therapy. However, extracranially secreted GHRH, as a mature or a truncated polypeptide, is often biologically active (Thomer et al., 1984) and a low level of serum GHRH (100 pg/ml) stimulates GH secretion (Corpas et al., 1993). These characteristics make GHRH an excellent candidate for plasmid mediated supplementation of a gene product.

Gene Delivery and in vivo Expression: Recently, the delivery of specific genes to somatic tissue in a manner that can correct inborn or acquired deficiencies and imbalances was proved to be possible. Gene-based drug delivery offers a number of advantages over the administration of recombinant proteins. These advantages include the conservation of native protein structure, improved biological activity, avoidance of systemic toxicities, and avoidance of infectious and toxic impurities. In addition, gene therapy allows for prolonged exposure to the protein in the therapeutic range, because the protein is synthesized and secreted continuously into the circulation.

The primary limitation of using recombinant protein is the limited availability of protein after each administration. Gene therapy using injectable DNA plasmid vectors overcomes this, because a single injection into the patient's skeletal muscle permits physiologic expression of the protein for extensive periods of time (WO 99/05300 and WO 01/06988). Injection of the vectors promotes the production of enzymes and hormones in animals in a manner that more closely mimics the natural process. Furthermore, among the non-viral techniques for gene transfer in vivo, the direct injection of plasmid DNA into muscle tissue is simple, inexpensive, and safe.

Direct plasmid DNA transfer is currently the basis of many emerging therapeutic strategies, as it avoids the potential problems associated with viral genes or lipid particles (Muramatsu et al., 1998). Skeletal muscle is a preferred target tissue, because the muscle fiber has a long life span and can be transduced by circular DNA plasmids. Skeletal muscle borne plasmids have been expressed efficiently over months or years in immunocompetent hosts (Davis et al., 1993; Tripathy et al., 1996). Plasmid DNA constructs are attractive candidate for direct therapy into the subjects skeletal muscle because they are well-defined entities, which are biochemically stable and have been used successfully for many years (Acsadi et al., 1991; Wolff et al., 1990). The relatively low expression levels, achieved after direct plasmid DNA injection are sometimes sufficient to prove bio-activity of secreted peptides (Danko and Wolff, 1994; Tsurumi et al., 1996).

Previously, we reported that in mice, human GHRH cDNA could be delivered to muscle by an injectable myogenic expression vector, where it transiently stimulated GH secretion over a period of two weeks (Draghia-Akli et al., 1997). We have then optimized this injectable vector system by incorporating a powerful synthetic muscle promoter (Li et al., 1999) coupled with a novel protease-resistant GHRH molecule with a substantially longer half-life and greater GH secretory activity (pSP-HV-GHRH) (Draghia-Akli et al., 1999). We improved vector delivery to skeletal muscle via a highly efficient electroporation technology (Wang et al., 1998). Using this combination of vector design and electric pulses plasmid delivery method, we were able to show increased growth and favorably modified body composition in pigs (Draghia-Akli et al., 1999) and rodents (Draghia-Akli et al., 2002). The current invention describes a new protease resistant, super-active GHRH analog, called TI-GHRH.

### SUMMARY

One aspect of the current invention is a composition for a modified growth hormone releasing hormone (“GHRH”) or functional biological equivalent thereof. A preferred embodiment of this invention includes a peptide with a general formula (-A₁-A₂-DAIFTNSYRKVL-A₃-QLSARKILQDI-A₄-A₅-RQQGERNQEQGA-OH), wherein A₁ is a D-or L-isomer of the amino acid tyrosine (“Y”), or histidine (“H”); A₂ is a D-or L-isomer of the amino acid alanine (“A”), valine (“V”), or isoleucine (“I”); A₃ is a D-or L-isomer of the amino acid alanine (“A”) or glycine (“G”); A₄ is a D-or L-isomer of the amino acid methionine (“M”), or leucine (“L”); A₅ is a D-or L-isomer of the amino acid serine (“S”) or asparagine (“N”). Other preferred embodiments include a modified GHRH or functional biological equivalent thereof as shown in Seq ID #3, Seq ID #4, and Seq ID #5. The modified GHRH can be defined as a biologically active polypeptide that was engineered to contain a distinct amino acid sequence while simultaneously having similar or improved biologically activity compared to the wild-type GHRH (“wt-GHRH”) polypeptide. One benefit of the claimed invention occurs when the modified GHRH composition is delivered to a subject. The modified GHRH increases the level of growth hormone (“GH”) secretion in a subject. The preferred subject is a domesticated animal or human. Other benefits outlined in preferred embodiments include: increased insulin-like growth factor I (“IGF-I”), increased weight gain; increased bone density, increases body length, improved immune function and improved cardiac function in aging mammals. Additionally, the modified GHRH composition is resistant to degradation when compared to the wt-GHRH.

Another aspect of the current invention is a nucleic acid molecule (e.g. DNA or RNA) that encodes the modified growth hormone releasing hormone (“GHRH”) or functional biological equivalent thereof. In a preferred embodiment, nucleic acid molecule further comprises a synthetic mammalian expression plasmid with a synthetic or eukaryotic promoter; and a poly adenelation signal; a selectable marker gene promoter; a ribosomal binding site; and an origin of replication. The synthetic or eukaryotic promoter, the nucleic acid sequence encoding the modified GHRH or functional biological equivalent thereof, and the poly-adenylation signal comprise therapeutic elements of the synthetic mammalian expression plasmid. The therapeutic elements are operatively linked and located in a first operatively-linked arrangement. Similarly, the selectable marker gene promoter, the ribosomal binding site, the selectable marker gene sequence, and the origin of replication comprise replication elements of the synthetic mammalian expression plasmid; the replication elements are operatively linked and located in a second operatively-linked arrangement. The first-operatively-linked arrangement and the second-operatively-linked arrangement comprise a circular structure of the synthetic mammalian expression plasmid. In a preferred embodiment, the synthetic mammalian expression plasmid is utilized for plasmid mediated gene supplementation. Other preferred embodiments of a nucleic acid molecule that encodes a modified GHRH comprise a 3′ untranslated region (“UTR”) encompassing a portion of a human growth hormone 3′UTR, and a modified myogenic promoter (e.g. pSPc5-12).

In preferred embodiments, the nucleic acid molecule that encodes the modified GHRH or functional biological equivalent thereof is combined with a transfection-facilitating polypeptide (e.g. poly-L-glutamate) for delivering the composition into a muscle cell of a subject. The encoded modified GHRH or functional biological equivalent thereof is expressed in a tissue specific manner in the subject.

### BRIEF DESCRIPTION OF FIGURES

FIG. 1 shows the amino acid sequence for porcine wild type growth hormone releasing hormone (“GHRH”) and a protease resistant TI-GHRH analog with extended activity that can increase GH secretory activity and stability;

FIG. 2 shows a the release of growth hormone (“GH”) in porcine primary pituitary culture that were simulated by different GHRH species isolated from conditioned media of skeletal muscle cells transfected with myogenic expression vectors driving porcine GHRH analogs; the analogs are denoted as follows: porcine wild type GHRH (1-40)OH (“pwt”); pGHRH with amino acid substitutions of Gly15 to Ala, Met27 to Leu and Ser28 to Asn is represented by (“15/27/28”); the 15/27/28 construct plus the conversion of Ala2 to Ile2 is represented by (“TI-GHRH”); the 15/27/28 construct plus the conversion of Ala2 to Val2 is represented by (“TV-GHRH”); the 15/27/28 construct plus conversion of Tyr1 with His, and Ala2 with Val is represented by (“HV-GHRH”); a construct coding for E.coli beta-galactosidase(“βgal”) is used as a negative control; and a positive control of recombinant human (“10 ng GHRH”);

FIG. 3 shows the enhanced stability of the TI-GHRH compared with wild type porcine GHRH over a 6 hour incubation in plasma;

FIG. 4 shows a schematic representation of three plasmid constructs: the porcine wild type (“pGHRH”); the TI-GHRH; and the β-galactosidase construct; all contain the SPc5-12 synthetic promoter and the 3′ untranslated region (“UTR”) of growth hormone (“GH”), kan (kanamycin resistance gene for bacterial selection), and neo (neomycin resistance gene for in vivo selection);

FIG. 5 shows the relative levels of serum IGF-I concentration in pSP-GHRH injected mice versus placebo injected mice that were exposed to a single injection of any one of the super analog GHRH myogenic expression vectors;

FIG. 6 shows the average body weight in mice after pSP-GHRH analogs were injected intra-muscularly compared to controls;

FIG. 7 shows the average lean body mass increase in mice after pSP-GHRH analogs were injected intramuscularly compared to controls;

FIG. 8 shows the average bone area increase in mice after pSP-GHRH analogs were injected intramuscularly compared to controls;

FIG. 9 shows the average body length increase in mice after TI-GHRH analogs were injected intramuscularly compared to controls;

FIG. 10 shows the average tibia length in mice after TI-GHRH analogs were injected intra-muscularly compared to controls;

FIG. 11 shows the average spleen weight in mice after TI-GHRH analogs were injected intramuscularly compared to controls.

FIG. 12 shows the E peak early filling velocity of mice hear after TI-GHRH analogs were injected intramuscularly compared with controls. Heart rate and A peak velocity are shown as control measures.

### DETAILED DESCRIPTION OF PREFERRED EMBODIMENTS

Terms:

The term “analog” as used herein includes any mutant of GHRH, or synthetic or naturally occurring peptide fragments of GHRH.

The term “codon” as used herein refers to any group of three consecutive nucleotide bases in a given messenger RNA molecule, or coding strand of DNA that specifies a particular amino-acid, or a starting or stopping signal for translation. The term codon also refers to base triplets in a DNA strand.

The term “coding region” as used herein refers to any portion of the DNA sequence that is transcribed into messenger RNA (mRNA) and then translated into a sequence of amino acids characteristic of a specific polypeptide.

The term “delivery” as used herein is defined as a means of introducing a material into a subject, a cell or any recipient, by means of chemical or biological process, injection, mixing, electroporation, sonoporation, or combination thereof, either without or under pressure.

The term “encoded GHRH” as used herein is a biologically active polypeptide.

The term “functional biological equivalent” of GHRH as used herein is a polypeptide that has been engineered to contain a distinct amino acid sequence while simultaneously having similar or improved biological activity when compared to the GHRH polypeptide.

The term “growth hormone” (“GH”) as used herein is defined as a hormone that relates to growth and acts as a chemical messenger to exert its action on a target cell.

The term “growth hormone releasing hormone” (“GHRH”) as used herein is defined as a hormone that facilitates or stimulates release of growth hormone, and to a lesser extent other pituitary hormones, such as prolactin.

The term “heterologous nucleic acid sequence” as used herein is defined as a DNA sequence consisting of differing regulatory and expression elements.

The term “modified GHRH” as used herein is a polypeptide that has been engineered to contain an amino acid sequence that is distinct from the wild-type GHRH polypeptide while simultaneously having similar or improved biologically activity when compared to the wild-type GHRH polypeptide. The wild-type GHRH polypeptide is the naturally occurring species-specific GHRH polypeptide of a subject, a cell or any recipient of the modified GHRH.

The term “nucleic acid expression construct” as used herein refers to any type of genetic construct comprising a nucleic acid coding for a RNA capable of being transcribed. The transcribed RNA is then capable of being translated into a peptide, polypeptide, or protein. The term “expression vector” or “expression plasmid” can also be used interchangeably.

The term “subject” as used herein refers to any species of the animal kingdom. In preferred embodiments it refers more specifically to humans and domesticated animals.

The term “domesticated animal” as used herein refers to animals used for: pets (e.g. cats, dogs, etc.); work (e.g. horses, cows, etc.); food (chicken, fish, lambs, pigs, etc); and all others known in the art.

The term “operatively linked” as used herein refers to elements or structures in a nucleic acid sequence that are linked by operative ability and not physical location. The elements or structures are capable of, or characterized by accomplishing a desired operation. It is recognized by one of ordinary skill in the art that it is not necessary for elements or structures in a nucleic acid sequence to be in a tandem or adjacent order to be operatively linked.

The term “promoter” as used herein refers to a sequence of DNA that directs the transcription of a gene. A promoter may direct the transcription of a prokaryotic or eukaryotic gene. A promoter may be “inducible,” initiating transcription in response to an inducing agent or, in contrast, a promoter may be “constitutive,” whereby an inducing agent does not regulate the rate of transcription. A promoter may be regulated in a tissue-specific or tissue-preferred manner, such that it is only active in transcribing the operable linked coding region in a specific tissue type or types.

The term “replication element” as used herein comprises nucleic acid sequences that will lead to replication of a plasmid in a specified host. One skilled in the art of molecular biology will recognize that the replication element may include, but is not limited to, a selectable marker gene promoter, a ribosomal binding site, a selectable marker gene sequence, and an origin of replication.

The term “therapeutic element” as used herein comprises nucleic acid sequences that will lead to an in vivo expression of an encoded gene product. One skilled in the art of molecular biology will recognize that the therapeutic element may include, but is not limited to a promoter sequence, a poly [A] sequence, or a 3′ or 5′ UTR.

The term “vector” as used herein refers to any vehicle that delivers a nucleic acid into a cell or organism. Examples include plasmid vectors, viral vectors, liposomes, or cationic lipids.

The standard one and three letter abbreviations for amino acids used herein are as follows: Alanine, A ala; Arginine, R, arg; Asparagine, N, asn; Aspartic acid, D, asp; Cysteine, C, cys; Glutamine, Q, gln; Glutamic acid, E, glu; Glycine, G, gly; Histidine, H, his; Isoleucine, I, ile; Leucine, L, leu; Lysine, K, lys; Methionine, M, met; Phenylalanine, F, phe; Proline, P, pro; Serine, S, ser; Threonine, T, thr; Tryptophan, W, trp; Tyrosine, Y, tyr; Valine, V, val.

In a preferred embodiment, the nucleic acid construct or vector of the present invention is a plasmid which comprises a synthetic myogenic (muscle-specific) promoter, a synthetic nucleotide sequence encoding a modified growth hormone releasing hormone (GHRH) or its analog, and a 3′ untranslated region (3′UTR).

Promoters and Enhancers. A “promoter” is a control sequence that is a region of a nucleic acid sequence at which the initiation and rate of transcription are controlled. It may contain genetic elements where regulatory proteins and molecules may bind such as RNA polymerase and transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one of naturally-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™. Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. In a specific embodiment the promoter is a synthetic myogenic promoter.

The identity of tissue-specific promoters or elements, as well as assays to characterize their activity, is well known to those of skill in the art. Examples of such regions include the human LIMK2 gene, the somatostatin receptor 2 gene, murine epididymal retinoic acid-binding gene, human CD4, mouse alpha2 (XI) collagen, DIA dopamine receptor gene, insulin-like growth factor II, human platelet endothelial cell adhesion molecule-1.

Initiation Signals and Internal Ribosome Binding Sites. A specific initiation signal also may be required for efficient translation (synthesis of the encoded protein) of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (“IES”) elements are used to create multigene, or polycistronic messages. IRES elements are able to bypass the ribosome scanning model of 5′ methylated Cap-dependent translation and begin translation at internal sites. IRES elements from two members of the picornavirus family (polio and encephalomyocarditis) have been described, as well an IES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Multiple Cloning Sites. Vectors can include a multiple cloning site (“MCS”), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Splicing Sites. Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression.

Polyadenylation Signals. In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the bovine or human growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

Origins of Replication. In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

Selectable and Screenable Markers. In certain embodiments of the invention, the cells that contain the nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker, such as the antibiotic resistance gene on the plasmid constructs (such as kanamycin, ampicylin, gentamycin, tetracycline, or chloramphenicol).

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

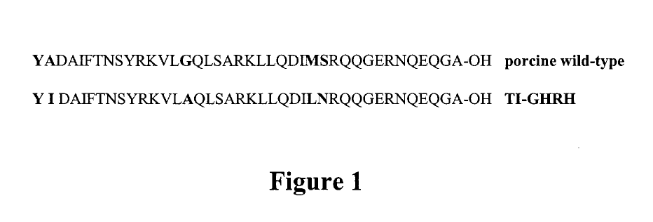

Growth hormone releasing hormone: Growth hormone releasing hormone (“GHRH”) has a short half-life in the circulatory system, both in humans and pigs (Frohman et al., 1984). However, by employing GHRH analogs with a prolonged biological half-life and/or improved secretagogue activity, it was possible to achieve enhanced growth hormone (“GH”) secretion. Therefore, GHRH mutants were generated by site-directed mutagenesis of a porcine (1-40)OH form of the cDNA (Seq ID #1). The site directed mutagenesis altered several amino acid codons of the wild type porcine GHRH (Seq ID #2). TI-GHRH mutant composition is shown in FIG. 1 and (Seq ID #3). The substitution of Gly15 to Ala15 was used to increase α-helical conformation and amphiphilic structure that resulted in decreased cleavage by trypsin-like enzymes (Su et al., 1991). Also, GHRH analogs with the Ala15-substitution display a 4-5 times higher affinity for the GHRH receptor (Reiss et al., 1993). We substituted Met27, Ser28 with Leu27, Asn28 (Kubiak et al., 1989) in order to reduce loss of biological activity due to oxidation of the Met27, thus forming a triple amino acid substitution denoted as 15/27/28-GHRH (Seq ID #4). Dipeptidyl peptidase IV is the prime serum GHRH degradative enzyme (Martin et al., 1993). Lower affinity dipeptidase substrates were created by further substitutions of 15/27/28-GHRH, and converting Ala2 for Ile2 (TI-GHRH, T1I2A15L27N28) or for Val2 (TV-GHRH—Seq ID #5) or by converting Tyr1 and Ala2 with His1 and Val2 (HV-GHRH—Seq ID #6). The HV-GHRH super-analog was presented in the U.S. patent application Ser. No. 10/021,403 filed on Dec. 12, 2001 and titled “Administration of nuclic acid sequence to female animal to enhance growth in offspring” with Schwartz, et al., listed as inventors, and U.S. patent application Ser. No. 09/624,268 filed on Jul. 24, 2000, 2000 and titled “Super Active Porcine Growth Hormone Releasing Hormone Analog” with Schwartz, et al., listed as inventors.

The invention may be better understood with reference to the following examples, which are representative of some of the embodiments of the invention, and are not intended to limit the invention.

### EXAMPLE 1

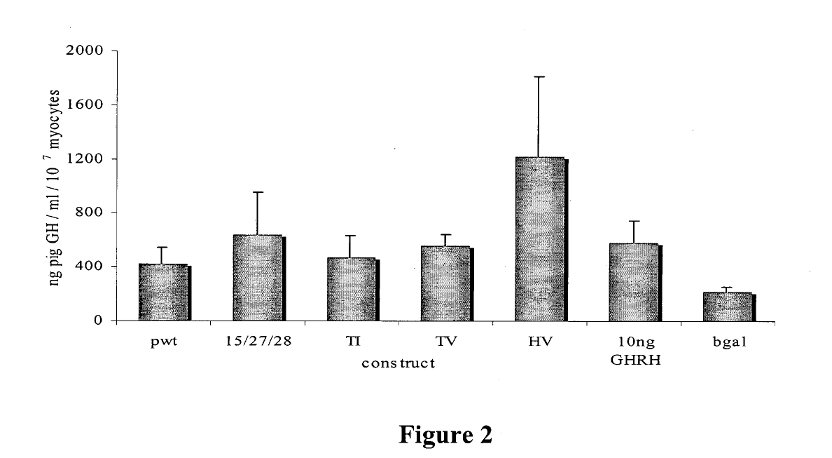

To test the biological potency of the mutated porcine GHRH (pGHRH) cDNA, a plasmid vector was engineered that could direct very high levels of skeletal muscle-specific gene expression with the use of a newly described synthetic muscle promoter, SPc5-12 (Li et al., 1999). A 228-bp fragment of pGHRH, which encodes the 31 amino acid signal peptide and a mature peptide pGHRH (Tyr1-Gly40) and/or the modified GHRH, or functional biological equivalents thereof, followed by the 3′ untranslated region of the human GH (“hGH”) cDNA. All expression vector elements were operatively linked and incorporated into myogenic GHRH expression vectors. Skeletal myoblasts were transfected with each construct. Purified GHRH moieties from conditioned culture media were assayed for potency by their ability to induce GH secretion in pig anterior pituitary cell cultures. As shown in FIG. 2, media were collected from the pituitary cell cultures after 24 hours and analyzed for porcine-specific GH by radioimmunoassay. The modified GHRH species (15/27/28-GHRH; TI-GHRH; TV-GHRH, HV-GHRH) showed 20% to 50% improvements in their capacity to stimulate GH secretion compared to wild-type porcine GHRH (“wt-GHRH”), as indicated by the increase in porcine GH levels from a baseline value of 200 ng/ml to 1600 ng/ml. Although not wanting to be bound by theory, the increase was probably produced by an increased affinity for the GHRH receptors present on the pituitary cells.

Experimental Cell culture conditions were as follows: The Minimal Essential Medium (“MEM”), heat-inactivated horse serum (“HIHS”), gentamycin, Hanks Balanced Salt Solution (“HBSS”), lipofectamine were obtained from Gibco BRL (Grand Island, N.Y.). Primary chicken myoblast cultures were obtained and transfected as previously described (Bergsma et al., 1986; Draghia-Akli et al., 1997). After transfection, the medium was changed to MEM which contained 2% HIHS to allow the cells to differentiate. Media and cells were harvested 72 hours post-differentiation. One day before harvesting, cells were washed twice in HBSS and the media changed to MEM, 0.1% bovine serum albumin (“BSA”). Conditioned media was treated by adding 0.25 volume of 1% triflouroacetic acid (“TFA”) and 1 mM phenylmethylsulfonylflouride (“PMSF”), frozen at −80° C., lyophilized, purified on C-18 Sep-Columns (Peninsula Laboratories, Belmont, Calif.), relyophilized and used in the radioimmuno assay (“RIA”) or resuspended in media conditioned for primary pig anterior pituitary culture. The pig anterior pituitary culture was obtained as previously described (Tanner et al., 1990).

Plasmid vectors containing the muscle specific synthetic promoter SPc5-12 were previously described (Li et al., 1999). Wild type and mutated porcine GHRH cDNAs were generated by site directed mutagenesis of GHRH cDNA (Altered Sites II in vitro Mutagenesis System, Promega, Madison, Wis.), and cloned into the BamHI/Hind III sites of pSPc5-12, to generate pSP-wt-GHRH, or pSP-TI-GHRH respectively. The 3′ untranslated region (3′UTR) of growth hormone was cloned downstream of GHRH cDNA. The resultant plasmids contained mutated coding region for GHRH, and the resultant amino acid sequences were not naturally present in mammals. Although not wanting to be bound by theory, the effects on treating GH deficient diseases is determined ultimately by the circulating levels of needed hormones. Several different plasmids that encode modified GHRH or functional biological equivalent thereof are as follows:

| | |
|---|---|
| Plasmid | Encoded Amino Acid Sequence |

| | |
|---|---|
| wt-GHRH | YADAIFTNSYRKVLGQLSARKLLQDIMSRQQGERNQE |
| | QGA-OH |
| HV-GHRH | HVDAIFTNSYRKVLAQLSARKLLQDILNRQQGERNQE |
| | QGA-OH |
| TI-GHRH | YIDAIFTNSYRKVLAQLSARKLLQDILNRQQGERNQE |
| | QGA-OH |
| TV-GHRH | YVDAIIFTNSYRKVLAQLSARKILQDILNRQQGERNQ |
| | EQGA-OH |
| 15/27/28GHRI | YADAIFTNSYRKVLAQLSARKLLQDILNRQQGERNQE |
| | QGA-OH |

In general, the encoded GHRH or functional biological equivalent thereof is of formula:

-A₋₁-A₂-DAIFTNSYRKVL-A₃-QLSARKLLQDI-A₄-A₅-RQQGERNQEQGA-OH

wherein: A₁ is a D-or L-isomer of an amino acid selected from the group consisting of tyrosine (“Y”), or histidine (“H”); A₂ is a D-or L-isomer of an amino acid selected from the group consisting of alanine (“A”), valine (“V”), or isoleucine (“I”); A₃ is a D-or L-isomer of an amino acid selected from the group consisting of alanine (“A”) or glycine (“G”); A₄ is a D-or L-isomer of an amino acid selected from the group consisting of methionine (“M”), or leucine (“L”); A₅ is a D-or L-isomer of an amino acid selected from the group consisting of serine (“S”) or asparagine (“N”).

Another plasmid that was utilized included the pSP-SEAP construct that contains the Sacl/HindIII SPc5-12 fragment, secreted embryonic alkaline phosphatase (SEAP) gene and SV40 3′UTR from pSEAP-2 Basic Vector (Clontech Laboratories, Inc., Palo Alto, Calif.).

The plasmids described above do not contain polylinker, IGF-I gene, a skeletal alpha-actin promoter or a skeletal alpha actin 3′ UTR/NCR. Furthermore, these plasmids were introduced by muscle injection, followed by in vivo electroporation, as described below.

In terms of “functional biological equivalents,” it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Functional biological equivalents are thus defined herein as those proteins (and polynucleotides) in selected amino acids (or codons) may be substituted. A peptide comprising a functional biological equivalent of GHRH is a polypeptide that has been engineered to contain distinct amino acid sequences while simultaneously having similar or improved biologically activity when compared to GHRH. For example one biological activity of GHRH is to facilitate growth hormone (“GH”) secretion in the subject.

### EXAMPLE 2

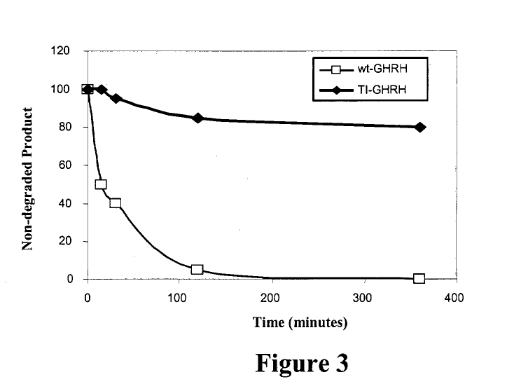

Stability of wild type GHRH and the analog TI-GHRH was then tested in porcine plasma by incubation of GHRH peptides in plasma, followed by solid phase extraction and HPLC analysis (Su et al., 1991). As shown in FIG. 3, at least 60% of the pGHRH was degraded within 30 minutes of incubation in plasma. In contrast, incubation of TI-GHRH in pig plasma for up to 6 hours showed that at least 70% of the polypeptide was protected against enzymatic cleavage indicating a considerable increase in the resistance of TI-GHRH to serum protease activity. No degradation was seen in the first 30 minutes of incubation. Plasma proteolytic activity on GHRH molecules. Chemically synthesized TI-GHRH was prepared by peptide synthesis. Briefly, pooled porcine plasma was collected from control pigs, and stored at −80° C. At the time of the test, the porcine plasma was thawed, centrifuged and allowed to equilibrate at 37° C. Mutant and wild-type GHRH samples were dissolved in the plasma sample to a final concentration of 300 μg/ml. Immediately after the addition of the GHRH, and 15, 30, 120 and 240 minutes later, 1 mL of plasma was withdrawn and acidified with 1 mL of 1M TFA. Acidified plasma was purified on C-18 affinity SEP-Pak columns, lyophilized, and analyzed by HPLC, using a Waters 600 multi-system delivery system, a Walters intelligent sample processor, type 717 and a Waters spectromonitor 490 (Walters Associates, Millipore Corp., Milford, Mass.). The mobile phase was (A) 0.1% TFA in H₂O, (B) 0.1% TFA in 95% ACN and 5% H₂O; the gradient was 80% (B) in 30 minutes. The flow rate was 0.75 mL/min. Detection was performed at 214 nm. The percent of peptide degraded at these time points was measured by integrated peak measurements.

### EXAMPLE 3

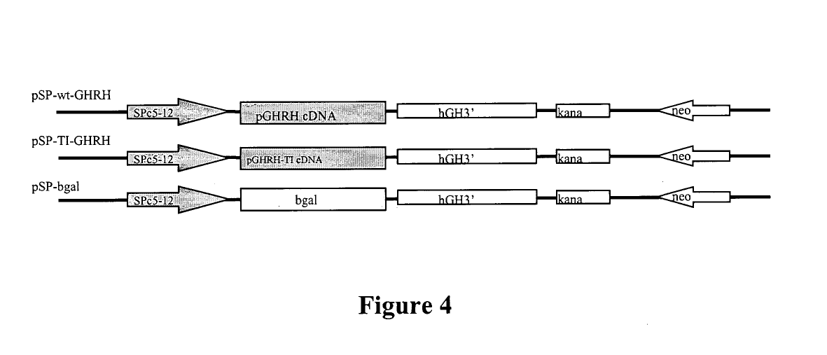

Biological activity of TI-GHRH in young animals. Muscle injection of pSP-TI-GHRH increases IGF-I serum levels over two months in treated mice. We asked if the optimized protease resistant pSP-TI-GHRH vector could affect in vivo, long-term expression of GHRH and stimulate secretion of GH and subsequently, IGF-I. Schematic maps of pSP-TI-GHRH, the wild type construct, pSP-wt-GHRH (positive control), and an E.coli. β-galactosidase expression vector, pSP-βal (placebo control), are shown in FIG. 4. Five weeks old SCID mice (immuno-deficient mice) were injected into the tibialis anterior muscle with 7.5 micrograms of one of the constructs. The injected muscle was placed within a caliper and electroporated, using optimized conditions of 200 V/cm with 6 pulses of 60 milliseconds, as described in Materials and Methods (Aihara and Miyazaki, 1998).

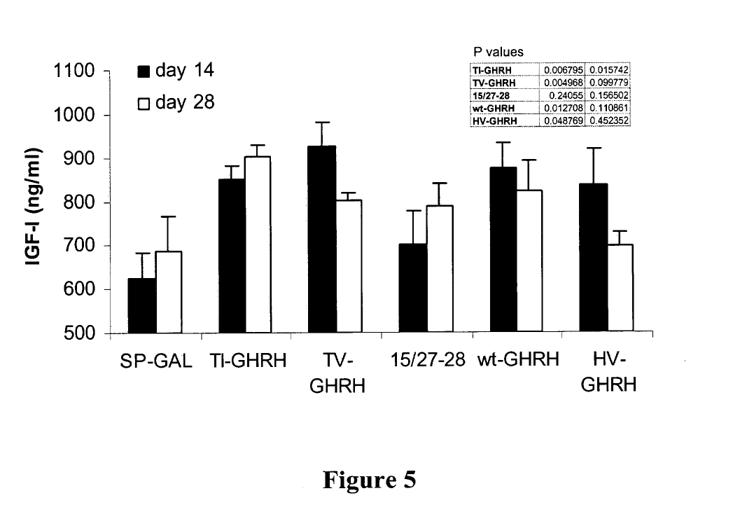

Animals were bled up to two month post-injection and serum was used for IGF-I measurements (FIG. 5). At 14 and 28 days post-injection, blood was collected and IGF-I levels were measured. All GHRH plasmid injected groups had highly significant increases in IGF-I levels compared to control animals, p<0.005. Some groups developed neutralizing antibodies, and in these cases the IGF-I levels dropped by the second time point. The animals injected with TI-GHRH did not develop any antibodies, and their modified TI-GHRH expression continued for two month, and correlated with significant changes in their body composition.

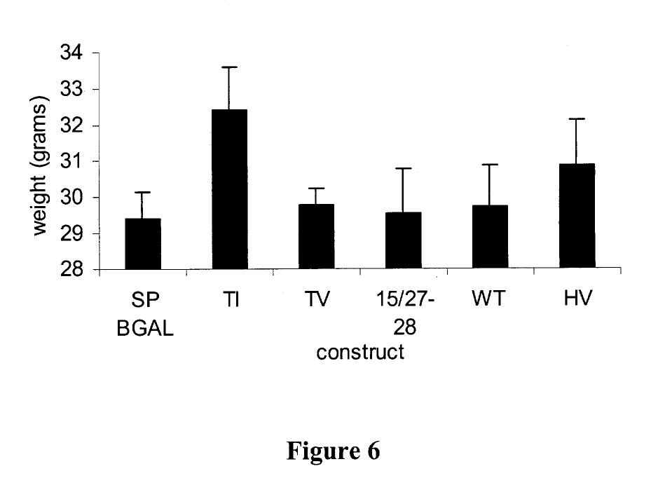

At the end of the experiment, body composition was performed in vivo, using a dual x-ray absortiometry technique (DEXA), with a high resolution scanner—PIXImus, and than post-mortem at necropsy. Blood was collected, centrifuged immediately at 4° C., and stored at −80° C. prior to analysis. Organs, carcass, fat from injected animals and controls were removed, weighed and snap frozen in liquid nitrogen. The TI-GHRH (p<0.03) and HV-GHRH injected animals were significantly heavier than controls at the end of the experiment (FIG. 6).

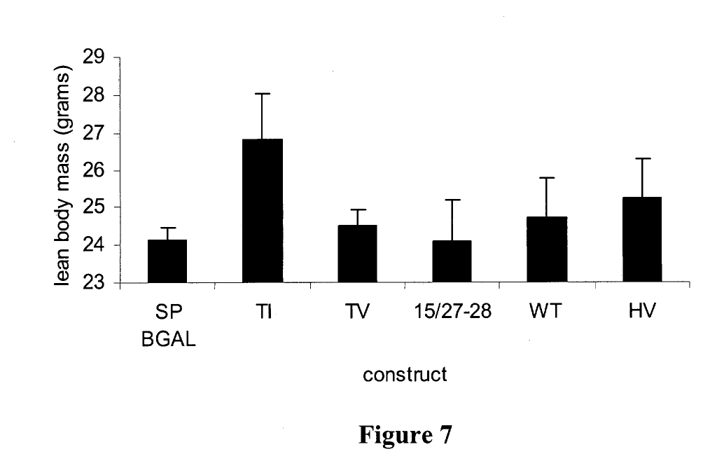

Body composition analysis by DEXA (total body fat, non-bone lean tissue mass and bone mineral area, content and density) showed significant changes in animals injected with the TI-GHRH plasmid. Non-bone lean body mass (FIG. 7) increased by 11% in TI-GHRH treated animals versus controls (p<0.036). The HV-GHRH injected animals had a significant increase in lean body mass of almost 5%, as seen in previous experiments.

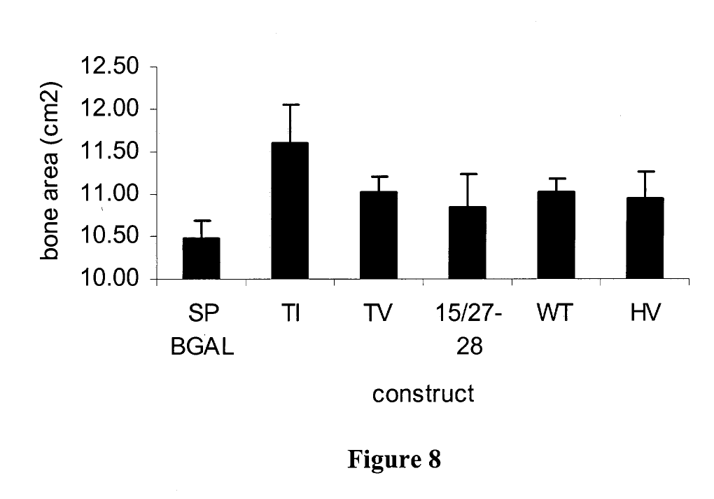

Upon injection with the TI-GHRH encoding plasmid, significant changes occurred in bone mineral area (FIG. 8), that increased by 10.7%, (p<0.027).

### EXAMPLE 4

A long-lasting therapy has the potential to replace classical GH therapy regimens and may stimulate the GH axis in a more physiologically appropriate manner. It is known that GHRH stimulates bone formation (Dubreuil et al., 1996), and our therapy may be used to promote post-fracture bone growth. Data show that GH plus IGF-I (delivered as recombinant proteins) synergistically increase lean muscle and body weight, total body weight, and were more effective in re-epithelialization of a burn wound than either GH or IGF-I alone (Meyer et al., 1996). Studies also showed that long-term stimulation of the GH axis, which includes doses in the range given to humans during clinical trials of GH deficiency and to revert age-related physiologic declines, has no overt deleterious effects on longevity and pathology in aged rodents (Kalu et al., 1998).

Using modified GHRH or functional biological equivalents thereof, it was possible to show that body composition was altered in treated animals. For example, twenty nine (“29”) month old mice were injected into the tibialis anterior muscle with 15 micrograms TI-GHRH plasmid. The control group received the pSP-βgal construct previously described. Injection followed by electroporation with calipers with standard conditions 200 V/cm, gap 5 mm on average, 3 pulses×2, 50 milliseconds/pulse. Cardiac function in these animals was evaluated by Doppler at day 0, 10 and 20 and echocardiogram at day 20. At the end of the experiment, body composition was performed post-mortem.

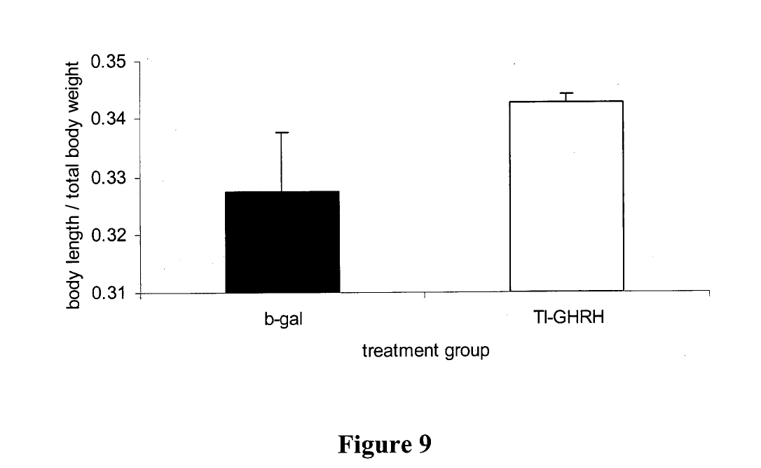

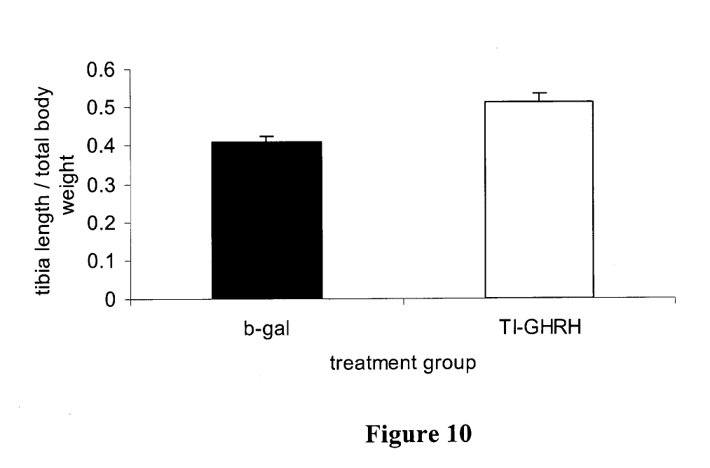

Body length had a tendency to be increased in treated animals, suggestive of axial growth (p<0.07) (FIG. 9). Furthermore, tibia length/total body weight was significantly increased in TI-GHRH animals (p<0.006) (FIG. 10), a sign of bone remodeling and growth in these extremely old animals.

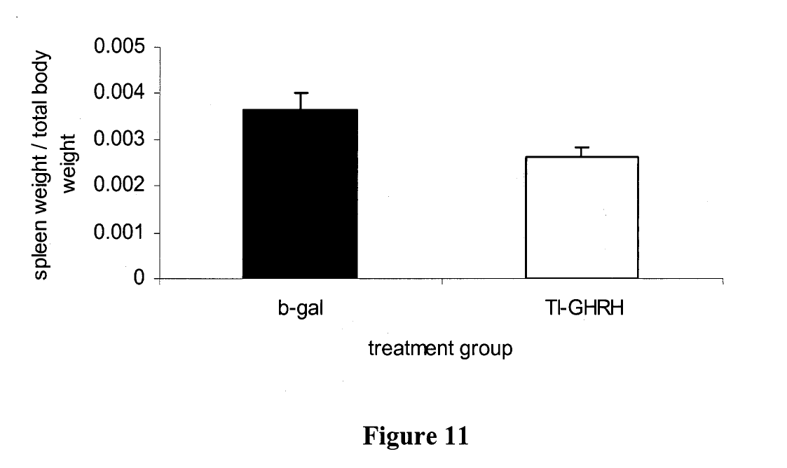

Aging, treated animals had reduced spleen weights/total body weight at necropsy (FIG. 11). Although not wanting to be bound by theory, this observed reduction was probably due to lymphocyte mobilization from the lymphatic organ to the general circulation. This change is sign of increased immune surveillance. Growth hormone (“GH”) is known to enhance immune responses, whether directly or through the IGF-I. GH secretagogues (“GHS”) have important effects on immunological functions in young and old mammals. In young mice, GHS cause a significant increase in peripheral blood lymphocyte counts. Old mice, treated with GHS for 3 weeks show significant resistance to the initiation of transplanted tumors and the subsequent metastases (Koo et al., 2001).

IGF-I also modulates the immune function, and has two major effects on B cell development: it acts as a differentiation factor to potentiate pro-B to pre-B cell maturation (Landreth et al., 1992), and it acts as a B cell proliferation cofactor to synergize with IL-7 (Landreth et al., 1985). There is evidence that macrophages are a rich source of IGF-I and that bone marrow stromal cells also produce IGF binding proteins (“IGFBP”) (Abboud et al., 1991). The treatment of mice with recombinant IGF-I increased the number of pre-B and mature B cells in bone marrow (Jardieu et al., 1994). The mature B cell remains sensitive to IGF-I as immunoglobulin production is also stimulated by IGF-I in vitro and in vivo (Robbins et al., 1994). The administration of recombinant IGF-I has been shown to increase the size of lymphoid organs in other species. In 1-yr-old sheep, an 8-week regimen of three daily injections of recombinant IGF-I increased spleen weight by 40% (Cottam et al., 1992). In the rabbit, cat, and dog similar effects of IGF-I have been observed. In the rhesus monkey, IGF-I also expands lymphocyte numbers (LeRoith et al., 1996). In blood, the percent CD4 cell count and the CD4/CD8 ratio falls with IGF-I treatment but were normalized by GH plus IGF-I. In the spleen combination treatment almost triples the percent CD4 cells and more than doubles the CD4/CD8 ratio. This paradox of differential effects on lymphocyte populations in different body compartments may be due to the anabolic hormones affecting lymphocyte trafficking as GH and IGF-I appears to cause lymphocytes to accumulate in lymphoid organs at the expense of lymphocyte numbers in the circulation (Clark et al., 1993; Jardieu et al., 1994). These observations in primates make it more likely that IGF-I will prove useful in other species such as dogs, cats or humans to improve immune function, especially after damage to the immune system or in immune senescence in the elderly (Auernhammer and Strasburger, 1995) or in patients with cancer.

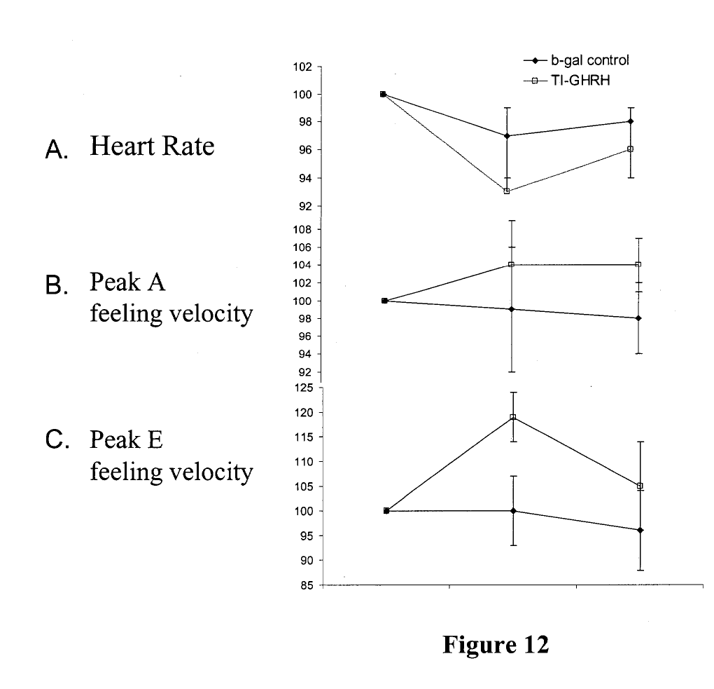

TI-GHRH treated animals also had increased cardiac function, as assessed by Doppler and echocardiogram. Heart rate is stable (within 5% throughout the assay) (FIG. 12A). Peak aortic flow velocity does not change either (FIG. 12B). This is unexpected as GH and IGF-I are supposed to induce eNOS and reduce peripheral vascular resistance. Altered diastolic filling is an important contributor to several cardiovascular disorders (Houlind et al., 2002; Richartz et al., 2002; Tang et al., 2002). Peak early filling velocity, a measure of the cardiac diastolic filling indices, increases impressively by 20% at 10 days post-treatment (FIG. 12C).

Among the non-viral techniques for plasmid transfer in vivo, the direct injection of plasmid DNA into muscle is simple, inexpensive, and safe. Applications of this methodology have been limited by the relatively low expression levels of the transferred DNA expression vectors. Previously, these levels have been insufficient to ensure systemic physiological levels of secreted proteins such as hormones, neurotrophic factors or coagulation factors in large mammals. Although not wanting to be bound by theory, in order to obtain growth of a large mammal by plasmid therapy, it is necessary to increase the potency of the myogenic vector system. The inventors recently described (Li et al., 1999) a strategy for the construction and the characterization of novel muscle synthetic promoters by the random assembly of E-boxes, MEF-2, TEF-1 and SRE sites. Several synthetic muscle promoters were identified whose transcriptional activity in terminally differentiated muscle greatly exceeded that of the natural myogenic skeletal α-actin gene promoter and viral promoters. Analysis of direct intramuscular injection of SPc5-12 driven DNA plasmid in normal mouse muscle revealed a 6-8-fold increase in activity over the ubiquitously expressed CMV promoter even after a month. As shown in FIG. 2, SPc5-12 was capable of eliciting moderate increases in growth and IGF-I levels by driving GHRH production in animals. Severe combined immunodeficient (SCID) adult male mice (aged 5-6 weeks at the beginning of the experiment) or NIH C57/Bl6 mice (aged 29 month) were housed and cared for in the animal facility of Baylor College of Medicine, Houston, Tex. Animals were maintained under environmental conditions of 10 h light/14 h darkness, in accordance with NIH Guidelines, USDA and Animal Welfare Act guidelines, and the protocol was approved by the Institutional Animal Care and Use Committee. On day 0, the animals were weighed and then, the left tibialis anterior muscle of mice was injected with plasmids in 25 μl PBS. The injection was followed by caliper electroporation, as previously described (Draghia-Akli et al., 1999). The animals were bled periodically, and serum was used to measure IGF-I levels. At the end of the experiment, body composition was performed in vivo, using the DEXA technique, and than at necropsy. Blood was collected, centrifuged immediately at 4° C., and stored at −80° C. prior to analysis. Organs, carcass, and fat from injected animals and controls were removed, weighed and snap frozen in liquid nitrogen.

Mouse IGF-I Radioimmunoassay: Mouse IGF-I was measured by heterologous, 100% cross-reacting rat radioimmunoassay. The sensitivity of the assay was 0.8 ng/ml; intra-assay and inter-assay variation was 3.4% and 4.5% respectively. The statistics and values shown in the figures are the mean±s.e.m. Specific p values were obtained by comparison using Students t-test or ANOVA analysis. A p<0.05 was set as the level of statistical significance.

Another significant improvement of our plasmid vector was the employment of a novel GHRH analog, TI-GHRH. Some individual amino acid substitutions leading to protease resistant GHRH molecules were previously tested in farm animals and humans (Frohman et al., 1989; Martin et al., 1993). The inventors have found that novel combination of five amino acid substitutions in the TI-GHRH construct resulted in increased GH secretagogue activity (as shown in assays on pig anterior pituitary somatotrophic cells) and was more resistant to serum proteases in vivo (FIG. 2).

### EXAMPLE 5

Although not wanting to be bound by theory, electro-plasmid therapy allows genes to be efficiently transferred and expressed in desired organs or tissues, and it may represent a new approach for highly effective plasmid supplementation therapy, that does not require viral genes or particles. The electroporation system was used previously in rodents and small animals and does not appear to cause significant distress. Electro-plasmid therapy increased transfection efficiency over 100-fold compared to classical plasmid therapy techniques and allowed for prolonged TI-GHRH expression.

Although not wanting to be bound by theory, enhanced biological potency and enhanced delivery protocols reduces the theoretical quantity of GHRH plasmid needed to achieve physiological levels of GH production and secretion. Treated mice did not experience any side effects from the therapy, had normal biochemical profiles, and with no associated pathology. The profound increases in IGF-I levels growth enhancement, the changes in body composition, increases in immune function and improved cardiac function indicate that ectopic expression of myogenic TI-GHRH vectors has the potential to replace classical GH therapy regimens and may stimulate the GH axis in a more physiologically appropriate manner. The TI-GHRH molecule, which displays a high degree of stability and GH secretory activity, may also be useful in human clinical medicine, since the serum proteases that degrade GHRH are similar in most mammals.

Hormones (e.g. GHRH and GH) often contain a complex feedback-regulated pathway, which are further complicated by chronic conditions such as cancer or AIDS. Without direct experimentation of GHRH or biological equivalents used in plasmid mediated supplementation, a beneficial therapy could not have been predicted by one skilled in the art to determine which modified GHRH or functional biological equitant thereof, encoded sequences will yield desired results. The invention described herein contains the compositions, descriptions, and results of essential experimentation that explored tissue specific and inducible regulation of distinctive nucleic acid sequences that encoded modified GHRH or biological equivalent thereof, which was not obvious based upon prior art.

One skilled in the art readily appreciates that the disclosed invention is well adapted to carry out the mentioned and inherent objectives. Growth hormone, growth hormone releasing hormone, modified growth hormone releasing hormone or functional biological equivalents, plasmids, vectors, pharmaceutical compositions, treatments, methods, procedures and techniques described herein are presented as representative of the preferred embodiments and are not intended as limitations of the scope of the invention. Thus, other uses will occur to those skilled in the art that are encompassed within the spirit and scope of the described invention.

The following documents and publications are incorporated by reference herein.

References Cited

U.S. Patent Documents

U.S. Pat. No. 5,847,066 issued on Dec. 8, 1998 with Coy et al. listed as inventors.

U.S. Pat. No. 5,846,936 issued on Dec. 8, 1998 with Felix et al. listed as inventors.

U.S. Pat. No. 5,792,747 issued on Aug. 11, 1998 with Schally et al. listed as inventors.

U.S. Pat. No. 5,776,901 issued on Jul. 7, 1998 with Bowers et al. listed as inventors.

U.S. Pat. No. 5,756,264 issued on May 26, 1998 with Schwartz et al. listed as inventors.

U.S. Pat. No. 5,696,089 issued on Dec. 9, 1997 with Felix et al. listed as inventors.

U.S. Pat. No. 5,486,505 issued on Jan. 23, 1996 with Bowers et al. listed as inventors.

U.S. Pat. No. 5,137,872 issued on Aug. 11, 1992 with Seely et al. listed as inventors.

U.S. Pat. No. 5,084,442 issued on Jan. 28, 1992 with Felix et al. listed as inventors.

U.S. Pat. No. 5,036,045 issued on Jul. 30, 1991 with Thorner listed as the inventor.

U.S. Pat. No. 5,023,322 issued on Jun. 11, 1991 with Kovacs et al. listed as inventors.

U.S. Pat. No. 4,839,344 issued on Jun. 13, 1989 with Bowers et al. listed as inventors.

U.S. Pat. No. 4,410,512 issued on Oct. 18, 1983 with Bowers et al. listed as inventors.

U.S. Pat. No. RE33,699 issued on Sep. 24, 1991 with Drengler listed as the inventor.

U.S. Pat. No. 4,833,166 issued on May 23, 1989 with Grosvenor et al. listed as inventors.

U.S. Pat. No. 4,228,158 issued on Oct. 14, 1980 with Momany et al. listed as inventors.

U.S. Pat. No. 4,228,156 issued on Oct. 14, 1980 with Momany et al. listed as inventors.

U.S. Pat. No. 4,226,857 issued on Oct. 7, 1980 with Momany et al. listed as inventors.

U.S. Pat. No. 4,224,316 issued on Sep. 23, 1980 with Momany et al. listed as inventors.

U.S. Pat. No. 4,223,021 issued on Sep. 16, 1980 with Momany et al. listed as inventors.

U.S. Pat. No. 4,223,020 issued on Sep. 16, 1980 with Momany et al. listed as inventors.

U.S. Pat. No. 4,223,019 issued on Sep. 16, 1980 with Momany et al. listed as inventors.

Publication Reference List

Abboud, S. L., C. R. Bethel, and D. C. Aron. 1991. Secretion of insulin like growth factor I and insulin-like growth factor-binding proteins by murine bone marrow stromal cells. J Clin. Invest 88:470-475.

Acsadi, G., G. Dickson, D. R. Love, A. Jani, F. S. Walsh, A. Gurusinghe, Wolff, J A, and K. E. Davies. 1991. Human dystrophin expression in mdx mice after intramuscular injection of DNA constructs. Nature 352:815-818.

Aihara, H. and J. Miyazaki. 1998. Gene transfer into muscle by electroporation in vivo. Nat. Biotechnol. 16:867-870.

Auernhammer, C. J. and C. J. Strasburger. 1995. Effects of growth hormone and insulin-like growth factor I on the immune system. European Journal of Endocrinology 133:635-645.

Bergsma, D. J., J. M. Grichnik, L. M. Gossett, and R. J. Schwartz. 1986. Delimitation and characterization of cis-acting DNA sequences required for the regulated expression and transcriptional control of the chicken skeletal alpha-actin gene. Molecular & Cellular Biology 6:2462-2475.

Butler, A. A., G. R. Ambler, B. H. Breier, D. LeRoith, C. T. Roberts, Jr., and P. D. Gluckman. 1994. Growth hormone (GH) and insulin-like growth factor-I (IGF-I) treatment of the GH-deficient dwarf rat: differential effects on IGF-I transcription start site expression in hepatic and extrahepatic tissues and lack of effect on type I IGF receptor mRNA expression. Mol. Cell Endocrinol. 101:321-330.

Caroni, P. and C. Schneider. 1994. Signaling by insulin-like growth factors in paralyzed skeletal muscle: rapid induction of IGF1 expression in muscle fibers and prevention of interstitial cell proliferation by IGF-BP5 and IGF-BP4. J. Neurosci. 14:3378-3388.

Clark, R., J. Strasser, S. McCabe, K. Robbins, and P. Jardieu. 1993. Insulin-like growth factor-1 stimulation of lymphopoiesis. J Clin. Invest 92:540-548.

Corpas, E., S. M. Harman, M. A. Pineyro, R. Roberson, and M. R. Blackman. 1993. Continuous subcutaneous infusions of growth hormone (GH) releasing hormone 1-44 for 14 days increase GH and insulin-like growth factor-I levels in old men. Journal of Clinical Endocrinology & Metabolism 76:134-138.

Cottam, Y. H., H. T. Blair, B. W. Gallaher, R. W. Purchas, B. H. Breier, S. N. McCutcheon, and P. D. Gluckman. 1992. Body growth, carcass composition, and endocrine changes in lambs chronically treated with recombinantly derived insulin-like growth factor-I. Endocrinology 130:2924-2930.

Danko, I. and J. A. Wolff. 1994. Direct gene transfer into muscle. Vaccine 12:1499-1502.

Davis, H. L., R. G. Whalen, and B. A. Demeneix. 1993. Direct gene transfer into skeletal muscle in vivo: factors affecting efficiency of transfer and stability of expression. Human Gene Therapy 4:151-159.

Draghia-Akli, R., M. L. Fiorotto, L. A. Hill, P. B. Malone, D. R. Deaver, and R. J. Schwartz. 1999. Myogenic expression of an injectable protease-resistant growth hormone-releasing hormone augments long-term growth in pigs. Nat. Biotechnol. 17:1179-1183.

Draghia-Akli, R., X. G. Li, R. J. Schwartz. 1997. Enhanced Growth By Ectopic Expression Of Growth Hormone Releasing Hormone Using An Injectable Myogenic Vector. nature biotechnology 15:1285-1289.

Draghia-Akli, R., P. B. Malone, L. A. Hill, K. M. Ellis, R. J. Schwartz, and J. L. Nordstrom. 2002. Enhanced animal growth via ligand-regulated GHRH myogenic-injectable vectors. FASEB J. 16:426-428.

Dubreuil, P., T. Abribat, B. Broxup, and P. Brazeau. 1996. Long-term growth hormone-releasing factor administration on growth hormone, insulin-like growth factor-I concentrations, and bone healing in the Beagle. Canadian Journal of Veterinary Research 60:7-13.

Etherton, T. D., J. P. Wiggins, C. S. Chung, C. M. Evock, J. F. Rebhun, and P. E. Walton. 1986. Stimulation of pig growth performance by porcine growth hormone and growth hormone-releasing factor. Journal of Animal Science 63:1389-1399.

Foncea, R., M. Andersson, A. Ketterman, V. Blakesley, M. Sapag-Hagar, P. H. Sugden, D. LeRoith, and S. Lavandero. 1997. Insulin-like growth factor-I rapidly activates multiple signal transduction pathways in cultured rat cardiac myocytes. J. Biol. Chem. 272:19115-19124.

Frohman, L. A., T. R. Downs, and P. Chomczynski. 1992. Regulation of growth hormone secretion. [Review]. Frontiers in Neuroendocrinology 13:344-405.

Frohman, L. A., T. R. Downs, E. P. Heimer, and A. M. Felix. 1989. Dipeptidylpeptidase IV and trypsin-like enzymatic degradation of human growth hormone-releasing hormone in plasma. J. Clin. Invest. 83:1533-1540.

Frohman, L. A., J. L. Thominet, C. B. Webb, M. L. Vance, H. Uderman, J. Rivier, W. Vale, and M. O. Thorner. 1984. Metabolic clearance and plasma disappearance rates of human pancreatic tumor growth hormone releasing factor in man. J. Clin. Invest. 73:1304-1311.

Gesundheit, N. and J. K. Alexander. 1995. Endocrine Therapy with Recombinant Hormones and Growth Factors. In: B. D. Weintraub (Ed.) Molecular Endocrinology: Basic Concepts and Clinical Correlations. pp. 491-507. Raven Press,Ltd., New York.

Hoess, R. H. and K. Abremski. 1985. Mechanism of strand cleavage and exchange in the Cre-lox site-specific recombination system. J. Mol. Biol. 181:351-362.

Houlind, K., A. P. Schroeder, H. Stodkilde-Jorgensen, P. K. Paulsen, H. Egeblad, and E. M. Pedersen. 2002. Intraventricular dispersion and temporal delay of early left ventricular filling after acute myocardial infarction. Assessment by magnetic resonance velocity mapping. Magn Reson. Imaging 20:249-260.

Jardieu, P., R. Clark, D. Mortensen, and K. Dorshkind. 1994. In vivo administration of insulin-like growth factor-I stimulates primary B lymphopoiesis and enhances lymphocyte recovery after bone marrow transplantation. J Immunol. 152:4320-4327.

Kalu, D. N., P. B. Orhii, C. Chen, D. Y. Lee, G. B. Hubbard, S. Lee, and Y. Olatunji-Bello. 1998. Aged-rodent models of long-term growth hormone therapy: lack of deleterious effect on longevity. J. Gerontol. A. Biol. Sci. Med. Sci. 53:B452-B463.

Koo, G. C., C. Huang, R. Camacho, C. Trainor, J. T. Blake, A. Sirotina-Meisher, K. D. Schleim, T. J. Wu, K. Cheng, R. Nargund, and G. McKissick. 2001. Immune enhancing effect of a growth hormone secretagogue. J Immunol. 166:4195-4201.

Kubiak, T. M., C. R. Kelly, and L. F. Krabill. 1989. In vitro metabolic degradation of a bovine growth hormone-releasing factor analog Leu27-bGRF(1-29)NH2 in bovine and porcine plasma. Correlation with plasma dipeptidylpeptidase activity. Drug Metabolism & Disposition 17:393-397.

Landreth, K. S., D. Engelhard, M. H. Beare, P. W. Kincade, N. Kapoor, and R. A. Good. 1985. Regulation of human B lymphopoiesis: effect of a urinary activity associated with cyclic neutropenia. J Immunol. 134:2305-2309.

Landreth, K. S., R. Narayanan, and K. Dorshkind. 1992. Insulin-like growth factor-I regulates pro-B cell differentiation. Blood 80:1207-1212.

Lapierre, H., G. Pelletier, D. Petitclerc, P. Dubreuil, J. Morisset, P. Gaudreau, Y. Couture, and P. Brazeau. 1991. Effect of human growth hormnone-releasing factor and(or) thyrotropin-releasing factor on growth, carcass composition, diet digestibility, nutrient balance, and plasma constituents in dairy calves. Journal of Animal Science 69:587-598.

LeRoith, D., J. Yanowski, E. P. Kaldjian, E. S. Jaffe, T. LeRoith, K. Purdue, B. D. Cooper, R. Pyle, and W. Adler. 1996. The effects of growth hormone and insulin-like growth factor I on the immune system of aged female monkeys. Endocrinology 137: 071-1079.

Li, X., E. M. Eastman, R. J. Schwartz, and R. Draghia-Akli. Synthetic muscle promoters: activities exceeding naturally occurring regulatory sequences. nature biotechnology 17[3], 241-245. 1999.

Ref Type: Generic

Liu, J. L. and D. LeRoith. 1999. Insulin-like growth factor I is essential for postnatal growth in response to growth hormone. Endocrinology 140:5178-5184.

Lowe, W. L., Jr., M. Adamo, H. Werner, C. T. Roberts, Jr., and D. LeRoith. 1989. Regulation by fasting of rat insulin-like growth factor I and its receptor. Effects on gene expression and binding. J. Clin. Invest 84:619-626.

Martin, R. A., D. L. Cleary, D. M. Guido, H. A. Zurcher-Neely, and T. M. Kubiak. 1993. Dipeptidyl peptidase IV (DPP-IV) from pig kidney cleaves analogs of bovine growth hormone-releasing factor (bGRF) modified at position 2 with Ser, Thr or Val. Extended DPP-IV substrate specificity? Biochimica et Biophysica Acta 1164:252-260.

Meyer, N. A., R. E. Barrow, and D. N. Herndon. 1996. Combined insulin-like growth factor-1 and growth hormone improves weight loss and wound healing in burned rats. J. Trauma 41:1008-1012.

Muramatsu, T., A. Nakamura, and H. M. Park. 1998. In vivo electroporation: A powerful and convenient means of nonviral gene transfer to tissues of living animals (Review) [In Process Citation]. Int. J. Mol. Med. 1:55-62.

Parks, J. S., R. W. Pfaffle, M. R. Brown, H. Abdul-Latif, and L. R. Meacham. 1995. Growth Hormone Deficiency. In: B. D. Weintraub (Ed.) Molecular Endocrinology: Basic Concepts and Clinical Correlations. pp. 473-490. Raven Press,Ltd., New York.

Parrizas, M. and D. LeRoith. 1997. Insulin-like growth factor-1 inhibition of apoptosis is associated with increased expression of the bcl-xL gene product. Endocrinology 138:1355-1358.

Rabinovsky, E. D., G. M. Smith, D. P. Browder, H. D. Shine, and J. L. McManaman. 1992. Peripheral nerve injury down-regulates CNTF expression in adult rat sciatic nerves. J. Neurosci. Res. 31:188-192.

Reiss, K., J. Kajstura, J. M. Capasso, T. A. Marino, and P. Anversa. 1993. Impairment of myocyte contractility following coronary artery narrowing is associated with activation of the myocyte IGF1 autocrine system, enhanced expression of late growth related genes, DNA synthesis, and myocyte nuclear mitotic division in rats. Exp. Cell Res. 207:348-360.

Richartz, B., G. Werner, M. Ferrari, and H. Figulla. 2002. Comparison of left ventricular systolic and diastolic function in patients with idiopathic dilated cardiomyopathy and mild heart failure versus those with severe heart failure. Am. J. Cardiol. 90:390.

Robbins, K., S. McCabe, T. Scheiner, J. Strasser, R. Clark, and P. Jardieu. 1994. Immunological effects of insulin-like growth factor-I—enhancement of immunoglobulin synthesis. Clin. Exp. Immunol. 95:337-342.

Su, C. M., L. R. Jensen, E. P. Heimer, A. M. Felix, Y. C. Pan, and T. F. Mowles. 1991. In vitro stability of growth hormone releasing factor (GRF) analogs in porcine plasma. Hormone & Metabolic Research 23:15-21.

Tang, W., D. K. Arnett, R. B. Devereux, L. D. Atwood, D. W. Kitzman, and D. C. Rao. 2002. Linkage of left ventricular early diastolic peak filling velocity to chromosome 5 in hypertensive African Americans: the HyperGEN echocardiography study. Am. J. Hypertens. 15:621-627.

Tanner, J. W., S. K. Davis, N. H. McArthur, J. T. French, and T. H. Welsh, Jr. 1990. Modulation of growth hormone (GH) secretion and GH mRNA levels by GH-releasing factor, somatostatin and secretagogues in cultured bovine adenohypophysial cells. J. Endocrinol. 125:109-115.

Thorner, M. O., L. A. Frohman, D. A. Leong, J. Thominet, T. Downs, P. Hellmann, J. Chitwood, J. M. Vaughan, and W. Vale. 1984. Extrahypothalamic growth-hormone-releasing factor (GRF) secretion is a rare cause of acromegaly: plasma GRF levels in 177 acromegalic patients. Journal of Clinical Endocrinology & Metabolism 59:846-849.

Tripathy, S. K., E. C. Svensson, H. B. Black, E. Goldwasser, M. Margalith, Hobart, P M, and J. M. Leiden. 1996. Long-term expression of erythropoietin in the systemic circulation of mice after intramuscular injection of a plasmid DNA vector. Proc. Natl. Acad. Sci. USA 93:10876-10880.

Tsurumi, Y., S. Takeshita, D. Chen, M. Kearney, S. T. Rossow, J. Passeri, J. R. Horowitz, J. F. Symes, and J. M. Isner. 1996. Direct intramuscular gene transfer of naked DNA encoding vascular endothelial growth factor augments collateral development and tissue perfusion [see comments]. Circulation 94:3281-3290.

van Rooij, E. M., B. L. Haagmans, H. L. Glansbeek, Y. E. de Visser, M. G. de Bruin, W. Boersma, and A. T. Bianchi. 2000. A DNA vaccine coding for glycoprotein B of pseudorabies virus induces cell-mediated immunity in pigs and reduces virus excretion early after infection. Vet. lnnmunol. Immunopathol. 74:121-136.

Veldhuis, J. D., A. Iranmanesh, and A. Weltman. 1997. Elements In The Pathophysiology Of Diminished Growth Hormone (GH) Secretion In Aging Humans. Endocrine 7:41-48.

Wang, L., W. Ma, R. Markovich, W. L. Lee, and P. H. Wang. 1998. Insulin-like growth factor I modulates induction of apoptotic signaling in H9C2 cardiac muscle cells. Endocrinology 139:1354-1360.

Wolff, J. A., R. W. Malone, P. Williams, W. Chong, G. Acsadi, A. Jani, Felgner, and P L. 1990. Direct gene transfer into mouse muscle in vivo. Science 247:1465-1468.

What is claimed is:

1. A composition comprising: a modified growth hormone releasing hormone (“GHRH”) or functional biological equivalent thereof with a general formula:
-A₋₁-A₂-DAIFTNSYRKVL-A₃-QLSARKLLQDI-A₄-A₅-RQQGERNQEQGA-OH
wherein: A₁ is a D-or L-isomer of the amino acid tyrosine (“Y”), or histidine (“H”); A₂ is a D-or L-isomer of the amino acid alanine (“A”), valine (“V”), or isoleucine (“I”); A₃ is a D-or L-isomer of the amino acid alanine (“A”) or glycine (“G”); A₄ is a D-or L-isomer of the amino acid methionine (“M”), or leucine (“L”); A₅ is a D-or L-isomer of the amino acid serine (“S”) or asparagine (“N”).

2. The composition of claim 1, wherein the modified GHRH or functional biological equivalent thereof is Seq ID #3.

3. The composition of claim 1, wherein the modified GHRH or functional biological equivalent thereof is Seq ID #4.

4. The composition of claim 1, wherein the modified GHRH or functional biological equivalent thereof is Seq ID #5.

5. The composition of claim 1, wherein the modified GHRH is a biologically active polypeptide; and the functional biological equivalent of GHRH is a polypeptide that has been engineered to contain a distinct amino acid sequence while simultaneously having similar or improved biologically activity when compared to a wild-type GHRH (“wt-GHRH”) polypeptide.

6. The composition of claim 5, wherein the modified GHRH or functional biological equivalent thereof increases growth hormone (“GH”) secretion when delivered into a subject.

7. The composition of claim 6, wherein the subject is a human or domesticated animal.

8. The composition of claim 5, wherein the modified GHRH or functional biological equivalent thereof increases a insulin-like growth factor I (“IGF-I”) secretion when delivered into a subject.

9. The composition of claim 5, wherein the modified GHRH or functional biological equivalent thereof improves cardiac function when delivered into a subject.

10. The composition of claim 5, wherein the modified GHRH or functional biological equivalent thereof increases weight gain when delivered into a subject.

11. The composition of claim 5, wherein the modified GHRH or functional biological equivalent thereof increases bone mineral density when the modified GHRH or functional biological equivalent thereof is delivered into a subject.

12. The composition of claim 5, wherein the modified GHRH or functional biological equivalent thereof increases body length, when the modified GHRH or functional biological equivalent thereof is delivered into a subject.

13. The composition of claim 5, wherein the modified GHRH or functional biological equivalent thereof improves immune function when the modified GHRH or functional biological equivalent thereof is delivered into a subject.

14. The composition of claim 5, wherein the modified GHRH or functional biological equivalent thereof improves cardiac function when the modified GHRH or functional biological equivalent thereof is delivered into a subject.

15. The composition of claim 5, wherein the modified GHRH or functional biological equivalent thereof is resistant to degradation when compared to the wt-GHRH.

16. The composition of claim 1, wherein the modified GHRH or functional biological equivalent thereof is translated from an encoded nucleic acid molecule, wherein the nucleic acid molecule is DNA or RNA.

17. The composition of claim 16, wherein the nucleic acid molecule further comprises a synthetic mammalian expression plasmid.

18. The composition of claim 17, wherein the synthetic mammalian expression plasmid further comprises:
a synthetic or eukaryotic promoter;
a poly-adenylation signal;
a selectable marker gene promoter;
a ribosomal binding site;
a selectable marker gene sequence; and
an origin of replication;
wherein the synthetic or eukaryotic promoter, the nucleic acid sequence encoding the modified GHRH or functional biological equivalent thereof, and the poly adenylation signal comprise therapeutic elements of the synthetic mammalian expression plasmid; the therapeutic elements are operatively linked and located in a first operatively-linked arrangement; the selectable marker gene promoter, the ribosomal binding site, the selectable marker gene sequence, and the origin of replication comprise replication elements of the synthetic mammalian expression plasmid; the replication elements are operatively linked and located in a second operatively-linked arrangement; the first-operatively-linked arrangement and the second-operatively-linked arrangement comprise a circular structure of the synthetic mammalian expression plasmid; and the synthetic mammalian expression plasmid is utilized for plasmid mediated gene supplementation.

19. The synthetic mammalian expression plasmid of claim 18, further comprising a 3′ untranslated region (“UTR”) operatively linked to the first operatively-linked arrangement.

20. The synthetic mammalian expression plasmid of claim 19, wherein the 3′ untranslated region (“UTR”) comprises a portion of a human growth hormone 3′ UTR.

21. The synthetic mammalian expression plasmid of claim 18, wherein the first operatively-linked arrangement comprises SeqID#7.

22. The synthetic mammalian expression plasmid of claim 18, wherein the first operatively-linked arrangement comprises SeqID#8.

23. The synthetic mammalian expression plasmid of claim 18, wherein the first operatively-linked arrangement comprises SeqID#9.

24. A nucleic acid expression construct encoding a modified growth hormone releasing hormone (“GHRH”) or functional biological equivalent thereof with a general formula:
-A₁-A₂-DAIFTNSYRKVL-A₃-QLSARKLLQDI-A₄-A₅-RQQGERNQEQGA-OH
wherein: A₁ is a D-or L-isomer of the amino acid tyrosine (“Y”), or histidine (“H”); A₂ is a D-or L-isomer of the amino acid alanine (“A”), valine (“V”), or isoleucine (“I”); A₃ is a D-or L-isomer of the amino acid alanine (“A”) or glycine (“G”); A₄ is a D-or L-isomer of the amino acid methionine (“M”), or leucine (“L”); A₅ is a D-or L-isomer of the amino acid serine (“S”) or asparagine (“N”), and wherein the modified GHRH.

25. The composition of claim 24, wherein the modified GHRH or functional biological equivalent thereof is Seq ID #3.

26. The composition of claim 24, wherein the modified GHRH or functional biological equivalent thereof is Seq ID #4.

27. The composition of claim 24, wherein the modified GHRH or functional biological equivalent thereof is Seq ID #5.

28. The composition of claim 24, wherein the nucleic acid expression construct further comprises, a transfection-facilitating polypeptide.

29. The composition of claim 28, wherein the transfection-facilitating polypeptide comprises a charged polypeptide.

30. The composition of claim 28, wherein the transfection-facilitating polypeptide comprises poly-L-glutamate.

31. The composition of claim 24, wherein the nucleic acid expression construct initiates expression of the encoded modified GHRH or functional biological equivalent thereof when delivered into a cell of a subject.

32. The composition of claim 31, wherein the encoded modified GHRH or functional biological equivalent thereof is capable of being expressed in a tissue specific cell of the subject.

33. The composition of claim 32, wherein the tissue specific cell of the subject comprises a muscle cell.

34. The composition of claim 31, wherein the subject is a human or domesticated animal.

35. The composition of claim 24, wherein the encoded modified GHRH is a biologically active polypeptide; and the encoded functional biological equivalent of GHRH is a polypeptide that has been engineered to contain a distinct amino acid sequence while simultaneously having similar or improved biologically activity when compared to the GHRH polypeptide.

36. The composition of claim 24, wherein the encoded modified GHRH or functional biological equivalent thereof facilitates growth hormone (“GH”) secretion when delivered into a subject.

37. The composition of claim 24, wherein the encoded modified GHRH or functional biological equivalent thereof increases a insulin-like growth factor I (“IGF-I”) secretion when delivered into a subject.

38. The composition of claim 24, wherein the encoded modified GHRH or functional biological equivalent thereof increases weight gain when delivered into a subject.

39. The composition of claim 24, wherein the encoded modified GHRH or functional biological equivalent thereof increases bone mineral density when the encoded modified GHRH or functional biological equivalent thereof is delivered into a subject.

40. The composition of claim 24, wherein the encoded modified GHRH or functional biological equivalent thereof increases body length when the modified GHRH or functional biological equivalent thereof is delivered into a subject.

41. The composition of claim 24, wherein the encoded modified GHRH or functional biological equivalent thereof improves immune function when the modified GHRH or functional biological equivalent thereof is delivered into a subject.

42. The composition of claim 24, wherein the encoded modified GHRH or functional biological equivalent thereof improves cardiac function when the modified GHRH or functional biological equivalent thereof is delivered into a subject.

43. The composition of claim 24, wherein the encoded modified GHRH or functional biological equivalent thereof is resistant to degradation when compared to the wt-GHRH.

44. A composition comprising: a modified GHRH of Seq ID #3.

45. The composition of claim 44, wherein the modified GHRH of Seq ID #3 is encoded by a nucleic acid expression construct.

46. The composition of claim 45, wherein the nucleic acid expression construct further comprises, a transfection-facilitating polypeptide.

47. The composition of claim 46, wherein the transfection-facilitating polypeptide comprises a charged polypeptide.

48. The composition of claim 46, wherein the transfection-facilitating polypeptide comprises poly-L-glutamate.

49. A composition comprising: a modified GHRH of Seq ID #4.

50. The composition of claim 49, wherein the modified GHRH of Seq ID #4 is encoded by a nucleic acid expression construct.

51. The composition of claim 50, wherein the nucleic acid expression construct further comprises, a transfection-facilitating polypeptide.

52. The composition of claim 51, wherein the transfection-facilitating polypeptide comprises a charged polypeptide.

53. The composition of claim 51, wherein the transfection-facilitating polypeptide comprises poly-L-glutamate.

54. A composition comprising: a modified GHRH of Seq ID #5.

55. The composition of claim 54, wherein the modified GHRH of Seq ID #5 is encoded by a nucleic acid expression construct.

56. The composition of claim 55, wherein the nucleic acid expression construct further comprises, a transfection-facilitating polypeptide.

57. The composition of claim 56, wherein the transfection-facilitating polypeptide comprises a charged polypeptide.

58. The composition of claim 56, wherein the transfection-facilitating polypeptide comprises poly-L-glutamate.

59. A method of increasing growth hormone (“GH”) secretion in a subject comprising: delivering into a cell of the subject a nucleic acid expression construct that encodes a modified growth-hormone-releasing-hormone (“GHRH”) or functional biological equivalent thereof, wherein the modified GHRH or functional biological equivalent thereof is of formula:
-A₋₁-A₂-DAIFTNSYRKVL-A₃-QLSARKLLQDI-A₄-A₅-RQQGERNQEQGA-OH
wherein: A₁ is a D-or L-isomer of the amino acid tyrosine (“Y”), or histidine (“H”); A₂ is a D-or L-isomer of the amino acid alanine (“A”), valine (“V”), or isoleucine (“I”); A₃ is a D-or L-isomer of the amino acid alanine (“A”) or glycine (“G”); A₄ is a D-or L-isomer of the amino acid methionine (“M”), or leucine (“L”); A₅ is a D-or L-isomer of the amino acid serine (“S”) or asparagine (“N”).

60. The method of claim 59, wherein delivering into the cell of the subject the nucleic acid expression construct is via electroporation.

61. The method of claim 59, wherein the cell of the subject is a somatic cell, a stem cell, or a germ cell.

62. The method of claim 59, wherein the cell of the subject is a muscle cell.

63. The method of claim 59, wherein the delivering into the cell of the subject the nucleic acid expression construct initiates expression of the encoded modified GHRH or functional biological equivalent thereof

64. The method of claim 59, wherein the modified GHRH or functional biological equivalent thereof is expressed in a tissue specific cell of the subject.

65. The method of claim 59, wherein the modified GHRH is a biologically active polypeptide; and the encoded functional biological equivalent of GHRH is a polypeptide that has been engineered to contain a distinct amino acid sequence while simultaneously having similar or improved biologically activity when compared to the GHRH polypeptide.

66. The method of claim 59, wherein the modified GHRH or functional biological equivalent thereof is Seq ID #3.

67. The method of claim 59, wherein the modified GHRH or functional biological equivalent thereof is Seq ID #4.

68. The method of claim 59, wherein the modified GHRH or functional biological equivalent thereof is Seq ID #5.

69. The method of claim 59, wherein the modified GHRH is a biologically active polypeptide; and the functional biological equivalent of GHRH is a polypeptide that has been engineered to contain a distinct amino acid sequence while simultaneously having similar or improved biologically activity when compared to a wild-type GHRH (“wt-GHRH”) polypeptide.

70. The method of claim 59, wherein the subject is a human or domesticated animal.

71. The method of claim 59, wherein the modified GHRII or functional biological equivalent thereof increases insulin-like growth factor I (“IGF-I”) secretion when delivered into a subject.

72. The method of claim 59, wherein the modified GHRH or functional biological equivalent thereof increases weight gain when delivered into a subject.

73. The method of claim 59, wherein the modified GHRH or functional biological equivalent thereof increases bone mineral density when the modified GHRH or functional biological equivalent thereof is delivered into a subject.

74. The method of claim 59, wherein the modified GHRH or functional biological equivalent thereof increases body length, when the modified GHRH or functional biological equivalent thereof is delivered into a subject.

75. The method of claim 59, wherein the modified GHRH or functional biological equivalent thereof improves the immune function when the modified GHRH or functional biological equivalent thereof is delivered into a subject.

76. The method of claim 59, wherein the modified GHRH or functional biological equivalent thereof improves cardiac function when the modified GHRH or functional biological equivalent thereof is delivered into a subject.

77. The method of claim 59, wherein the modified GHRH or functional biological equivalent thereof is resistant to degradation when compared to the wt-GHRH.

78. The method of claim 59, wherein the modified GHRH or functional biological equivalent thereof is resistant to degradation when compared to the wt-GHRH.


SEQUENCE LISTING (9 sequences)

SEQ ID NO: 1 - DNA, 219 bp, artificial sequence
  This is the cDNA for Porcine growth hormone releasing hormone
atggtgctctgggtgttcttctttgtgatcctcaccctcagcaacagctcccactgctccccacctccccctttgaccctcaggatgcggcggcacgtagatgccatcttcaccaacagctaccggaaggtgctggcccagctgtccgcccgcaagctgctccaggacatcctgaacaggcagcagggagagaggaaccaagagcaaggagcataatga

SEQ ID NO: 2 - protein, 40 aa, artificial sequence
  This is the amino acid sequence for procine growth hormone releasing hormone.
YADAIFTNSYRKVLGQLSARKLLQDIMSRQQGERNQEQGA

SEQ ID NO: 3 - protein, 40 aa, artificial sequence
  This is a modified amino acid sequence for growth hormone releasing hormone (GHRH).   alpha-helical confomation was increased by substituting Cly15 to Ala15.
YIDAIFTNSYRKVLAQLSARKLLQDILNRQQGERNQEQGA

SEQ ID NO: 4 - protein, 40 aa, artificial sequence
  This is a porcine growth hormone releasing hormone (“GHRH”) that has the following substitutions Met27, Ser28 with Leu27 and Asn28.
YADAIFTNSYRKVLAQLSARKLLQDILNRQQGERNQEQGA

SEQ ID NO: 5 - protein, 40 aa, artificial sequence
  This is a growth hormone releasing hormone that has a Val2 substitution for a Ile 2.
YVDAIFTNSYRKVLAQLSARKLLQDILNRQQGERNQEQGA

SEQ ID NO: 6 - protein, 40 aa, artificial sequence
  This is a growth hormone releasing hormone (“GHRH”) with a His1 and val2 substituting the Try1 and Ala2.
HVDAIFTNSYRKVLAQLSARKLLQDILNRQQGERNQEQGA

SEQ ID NO: 7 - DNA, 3534 bp, artificial sequence
  This is the nucleic acid sequence for the operatively linked components of the TI-GHRH plamsmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatatcgatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 8 - DNA, 3534 bp, artificial sequence; identical to SEQ ID NO: 7
  This is the nucleic acid sequence for the operatively linked components of the TV-GHRH plasmid.

SEQ ID NO: 9 - DNA, 3534 bp, artificial sequence; identical to SEQ ID NO: 7
  This is the nucleic acid sequence for the operatively linked components of the 15/27/28 GHRH plasmid.